(19) United States
(12) Patent Application Publication
(10) Pub. No.: US 2025/0061571 A1
(43) Pub. Date: Feb. 20, 2025

(54) Method and Apparatus for Acquiring a Position of a Region to be Examined for a Medical Imaging Apparatus

(71) Applicant: Siemens Healthineers AG, Forchheim (DE)

(72) Inventors: Johann Sukkau, Herzogenaurach (DE); Julian Wohlers, Erlangen (DE); Florian Meise, Hausen (DE)

(73) Assignee: Siemens Healthineers AG, Forchheim (DE)

(21) Appl. No.: 18/806,098

(22) Filed: Aug. 15, 2024

(30) Foreign Application Priority Data
Aug. 17, 2023 (EP) 23192000.0

(51) Int. Cl. G06T 7/00; A61B 90/00; G06T 7/11

(52) U.S. Cl. CPC G06T 7/0012; A61B 90/39; G06T 7/11; G06T 2207/10088

(57) ABSTRACT

In a method for acquiring a position of a region to be examined of an object for a medical imaging examination, a marker element is manually positioned at the region to be examined of the object, the position of the marker element is acquired by an acquisition apparatus, a feedback marking is projected at the acquired position of the marker element by a projection apparatus and based on a correction factor in the z-direction that is considered when ascertaining a projection position of the feedback marking, the acquisition of the position of the marker element and of the projection of the feedback marking is repeated until the feedback marking coincides with the position of the marker element, and the position of the marker element is defined as the position of the region to be examined of the object.

### CROSS REFERENCE TO RELATED APPLICATIONS

This patent application claims priority to European Patent Application No. 23192000.0, filed Aug. 17, 2023, which is incorporated herein by reference in its entirety.

### BACKGROUND

### Field

The present disclosure relates to a method for acquiring a position of a region to be examined of an object for a medical imaging examination. Furthermore, the present disclosure also relates to a medical imaging apparatus comprising an acquisition apparatus, which is embodied for acquiring a position of a marker element, a projection apparatus which is embodied to project a feedback marking at the acquired position of the marker element, a camera and a computing unit, wherein the medical imaging apparatus is embodied to carry out a method for acquiring a position of a region to be examined of an object for a medical imaging examination.

### Related Art

For a medical imaging examination, in particular a magnetic resonance examination, it is necessary for the patient, in particular a region to be examined of the patient, to be correctly positioned in the isocenter of the medical imaging apparatus, for example a magnetic resonance apparatus. For example, the region to be examined of the patient is marked with a laser beam which is arranged on a front side of a magnetic resonance apparatus. For this a couch, on which the patient is positioned for the magnetic resonance examination, has to be moved in the z-direction, which corresponds to the longitudinal direction of the couch, until the region to be examined of the patient coincides with the laser marking. Following the marking of the region to be examined the couch is automatically moved, together with the patient, into a recording region of the magnetic resonance apparatus, with the region to be examined or the marked region of the patient being positioned in the isocenter. A positioning of this kind is very difficult for an inexperienced and/or unpracticed user, however, so the positioning requires a great deal of time and/or incorrect positionings can also occur.

From DE 10 2021 202 978 A1 an apparatus for acquiring and marking a region to be examined of a patient is known, wherein the apparatus is arranged on a ceiling above the couch. In this connection, the region to be examined is marked by a user with a marker object and is acquired by an acquisition apparatus. A feedback marking at the acquired position is then projected by means of a projection apparatus. If the position of the feedback marking coincides with the position of the marker object a shift of the couch up to the isocenter is ascertained using this position.

If, however, the projection apparatus is permanently arranged on the ceiling of the examination space, a method of this kind has the drawback that projection distortions occur in the z-direction which are all the greater, the higher a profile of the patient at the projection site and the further away the to be examined is relative to the position of the projection apparatus.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a medical imaging apparatus with an acquisition apparatus and a projection apparatus according to the disclosure.

FIG. 2 shows a method for acquiring a position of a region to be examined of an object for a medical imaging examination according to the disclosure.

FIG. 3 shows a detailed view of the projection apparatus with the couch and an object positioned on the couch according to the disclosure.

FIG. 4 shows a projection of a marking line on the object without considering a correction factor and a projection of a marking line while considering the correction factor according to the disclosure.

The exemplary embodiments of the present disclosure will be described with reference to the accompanying drawings. Elements, features and components that are identical, functionally identical and have the same effect are—insofar as is not stated otherwise-respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the disclosure is to provide fast and exact marking of the region to be examined.

A method for acquiring a position of a region to be examined of an object for a medical imaging examination, may include:
positioning a marker element at the region to be examined of the object, wherein the marker element is positioned manually by a medical operator,
acquiring the position of the marker element by means of an acquisition apparatus,
projecting a feedback marking at the acquired position of the marker element by means of a projection apparatus, wherein a correction factor in the z-direction is considered when ascertaining a position of the feedback marking,
repeating the acquisition of the position of the marker element and of the projection of the feedback marking until the feedback marking coincides with the region of the object to be examined, and
defining the position of the marker element as the position of the region to be examined.

The medical imaging examination can be a computed tomography examination (CT examination), a PET examination (positron emission tomography examination), a magnetic resonance examination or also a combination of a plurality of medical imaging examinations, such as a magnetic resonance PET examination or a CT-PET examination. Accordingly, the medical imaging apparatus, with which a method of this kind is carried out, can also comprise a computed tomography apparatus (CT apparatus), a PET apparatus (positron emission tomography apparatus), a magnetic resonance apparatus or a combination of a plurality of medical imaging apparatuses, such as a magnetic resonance PET apparatus or a CT-PET apparatus.

The object to be examined may include a patient. In particular, a clinical and/diagnostic problem on the patient is to be clarified by means of the medical imaging examination. In addition, the object can also comprise a phantom for adjustment measurements, for example, and/or further objects that seem expedient to the person skilled in the art.

The region to be examined of the object, in particular of the patient, comprises that region for which a clinical and/diagnostic problem is to be clarified. For example, the region to be examined can comprise an organ, a joint, a head, etc. of a patient.

In order to acquire the region to be examined the user may position a marker element on the region to be examined of the object and/or the patient, with the marker element being identified as such by the acquisition apparatus and its position being acquired by means of the acquisition apparatus. The marker element can comprise, for example, a finger of the medical operator or also a hand-guided item, for example a marker bar. In an exemplary embodiment, a tip of the finger and/or a tip of the hand-guided marker element is defined as the position of the marker element. Alternatively, or additionally, the region to be examined can also be defined and/or marked on a display, for example on a touch display, by the medical operator.

The acquisition apparatus is embodied to acquire a position of the region to be examined of the object, in particular of the patient, for a pending medical imaging examination. The acquisition apparatus can comprise a camera and/or other image sensor(s), for example a 2D camera, such as at least two 2D cameras, or a 3D camera. The acquisition apparatus may comprise more than one camera, such as a camera array. The acquisition apparatus may be arranged on a ceiling of an examination space in which the medical imaging apparatus is arranged. Additionally, or alternatively, the acquisition apparatus may include one or more position sensors. In particular, the acquisition apparatus is arranged in a front region in front of a front side of the scanner unit (scanner). The front region adjoins the front side in front of the scanner unit. In addition, the acquisition apparatus can comprise further units and/or sensors for acquiring the region to be examined, which can be arranged, for example, on a couch of the medical imaging apparatus.

The projection apparatus may include a video projector and/or a laser marking unit which is embodied for a projection and/or display of a feedback marking at the position of the marker element acquired by the acquisition apparatus. The feedback marking may include a marking line whose z-position in the z-direction is defined by the marker element, with the marking line running in the x-direction. In an exemplary embodiment, the marking line runs in the x-direction over the entire width of the couch. The z-direction corresponds to the longitudinal direction (e.g., length) of the couch and/or an entry movement direction of the couch into the recording region. The x-direction (e.g. width of the couch) is oriented transversely to the z-direction in the horizontal direction. By means of the feedback marking the user receives feedback as to whether the region to be examined of the object and/or patient was correctly acquired. The projection apparatus may be arranged on the ceiling of the examination space. In particular, the projection apparatus may be arranged in a front region in front of a front side of the scanner unit (scanner).

Since the projection apparatus is permanently arranged on the ceiling of the examination space, a distortion occurs when the feedback marking is projected, which is all the more pronounced the further away the marker element is and therewith the region to be examined is from a position of the projection apparatus relative to the z-direction. In addition, the distortion is also dependent on a height (y-direction) of a profile of the object at the position of the marker element and therewith of the region to be examined. The correction factor corrects the distortion in the z-direction, so the feedback marking appears at the position of the marker element and therewith at the region to be examined.

The method steps of acquiring the position of the marker element and of the projection of the feedback marking are repeated until the feedback marking coincides with the position of the marker element and therewith the position of the region to be examined of the object. For example, after receiving the feedback marking, if this does not coincide with a position of the region to be examined of the object, in particular of the patient, by manually repositioning the marker element it is possible to communicate to the acquisition apparatus that the acquired position was not correct. By contrast, retaining the position of the marker element and/or complete removal of the marker element from the acquisition region of the acquisition apparatus can signal to the acquisition apparatus that acquisition of the position of the marker element is complete. As soon as the position of the feedback marking coincides with the position of the marker element and therewith of the region to be examined of the patient for the user and he signals to the acquisition apparatus that the position is correct, the position of the marker element is defined as the position of the region to be examined of the object.

The acquisition apparatus has for this purpose a computing unit and/or a controller with appropriate software which identifies the marker element from the image data acquired by means of the acquisition apparatus. The computing unit and/or controller of the acquisition apparatus is embodied to identify and determine a position and/or change in position of the marker element. Furthermore, the computing unit and/or the controller of the acquisition apparatus is embodied to identify termination of the acquisition process, for example by way of a retention of the position of the marker element and/or complete removal of the marker element from the acquisition region of the acquisition apparatus.

The inventive method is controlled by a computing unit of a medical imaging apparatus. The computing unit comprises at least one computing module and/or a processor, with the computing unit being embodied to control the method. The computing unit is thus embodied, in particular, to execute computer-readable instructions. In particular, the computing unit comprises a memory unit, with computer-readable items of information being stored on the memory unit, with the computing unit being embodied to load the computer-readable items of information from the memory unit and to execute the computer-readable items of information.

The components of the computing unit can be embodied for the most part in the form of software components. Basically, these components can, however, also be partially implemented, in particular if particularly fast calculations are involved, in the form of software-assisted hardware components, for example FPGAs or the like. Similarly, the required interfaces, for example if it is only a matter of incorporating data from other software components, can be embodied as software interfaces. However, they can also be embodied as interfaces constructed in terms of hardware, which are actuated by way of suitable software. Of course, it is also conceivable that a plurality of said components is implemented in a combined manner in the form of an individual software component or software-assisted hardware components.

In particular, the computing unit is embodied to appropriately control the projection apparatus such that it directs its projection region to the position of the marker element acquired by means of the acquisition apparatus. In particular, the computing unit is also embodied to determine the correction factor for the projection of the feedback marking and to already take it into account when transmitting the position information of the feedback marking to the projection apparatus. The projection apparatus can already receive a corrected position for the feedback marking. In addition, the computing unit can also control a communication between the acquisition apparatus and the projection apparatus.

The disclosure has the advantage that fast and exact marking of the region to be examined can be provided for a medical imaging examination. Furthermore, the correct and/or exact marking of the marker element, and therewith of the region to be examined of the object means, a correct positioning of the region to be examined inside of a patient-receiving region of a medical imaging apparatus can be achieved and therewith undesirable incorrect positionings can also be prevented. Medical imaging examinations can thereby also be carried out easily and quickly and repetitions of individual measurements owing to an incorrect positioning can be reduced and/or prevented. In particular, a medical operator can advantageously be assisted in this way in a workflow for positioning the region to be examined inside of the isocenter.

A further advantage is that for inputting and/or acquiring the region to be examined of the object it is not necessary for a user input to be made at an input unit, for example touching a touch display, so hygiene standards can be maintained particularly easily during a medical imaging examination.

In an advantageous development of the inventive method it can be provided that in order to ascertain the correction factor, 3D data of the object is acquired and a 3D profile of the object is determined using the 3D data. In an exemplary embodiment, the 3D data of the object is acquired by means of a camera. In an exemplary embodiment, the camera is incorporated by the acquisition apparatus. In an alternative embodiment the camera can also be embodied separately from the acquisition apparatus. The camera can comprise a 3D camera for acquiring the 3D profile. As an alternative to this at least two 2D cameras can also be used for acquiring the 3D profile. The computing unit of the medical imaging apparatus has appropriate evaluating software for determining a 3D profile of the object, in particular of a patient, from the 3D data. A height of the object (y-direction), in particular of the patient, which the patient has when lying on a reclining area of the patient table relative to the reclining area, can advantageously be determined from the 3D profile and be provided for ascertaining the correction factor.

In an advantageous development of the inventive method it can be provided that a height of the object at the acquired position of the marker element is ascertained from the 3D profile. The height of the object, in particular of the patient, at the acquired position of the marker element comprises an extent of the object, in particular of the patient, in the y-direction and/or vertical direction relative to a reclining area of the couch on which the object, in particular the patient, is arranged for a medical imaging examination. In an exemplary embodiment, the height of the object, in particular of the patient, is ascertained relative to the reclining area of the couch by means of the computing unit of the medical imaging apparatus. Ascertaining the height of the object, in particular of the patient, relative to the reclining area of the couch at the acquired position of the marker element enables particularly exact ascertainment of a correction factor which is considered when the feedback marking is projected.

In an advantageous development of the inventive method it can be provided that the feedback marking for defining the position of the region to be examined in the z-direction comprises a projected marking line in the x-direction on the object, with a height of the object being ascertained from the 3D profile of the object for each position in the x-direction of the marking line. In this way a correction factor can be ascertained and/or provided for each position x-direction. The individual correction factors can be different for different positions in the x-direction. This also makes it possible for the marking line to mark a straight line in the x-direction for the medical operator independently of the profile of the object, in particular of the patient.

In an advantageous development of the inventive method it can be provided that the correction factor in the z-direction comprises a height (in the y-direction) of the object at the acquired position of the marker element. In an exemplary embodiment, correction factor comprises a height of the object (in the y-direction), in particular of the patient, for each position in the x-direction. This makes it possible for the marking line to mark a straight line in the x-direction for the medical operator independently of the profile of the object, in particular of the patient.

In an advantageous development of the inventive method it can be provided that the correction factor comprises a spacing of the marker element in the z-direction in respect of the position of the projection apparatus and a height of the projection apparatus above the couch. The correction factor for each position in the x-direction may be calculated as follows:

$${KF}_{x} = \frac{d}{C} {H}_{x}$$

Here KFₓ is the correction factor and/or a correction value at the position x, d is the spacing of the acquired position of the marker element in the z-direction. C comprises the height of the projection apparatus above the couch (in the y-direction). Hₓ is the height of the object (in the y-direction), in particular of the patient, at the position x relative to the reclining area of the couch. The correction factor for each position in the x-direction at the position of the marker element may be ascertained by means of the computing unit of the medical imaging apparatus. In this way the correction factor can be ascertained particularly exactly and in a profile- and/or height-dependent manner for each position in the x-direction. In particular, a misinterpretation of the feedback marking can be prevented in this way and therewith also an incorrect positioning for the medical imaging examination.

In an advantageous development of the inventive method it can be provided that an eye area of the patient is ascertained using 3D data of a patient, wherein the eye area of the patient is excluded from a projection region of the projection apparatus when the feedback marking is projected. The projection region of the projection apparatus may comprise a region on the object at which the feedback marking is projected. In particular, the eye area of the patient is ascertained from the 3D data by means of the computing unit of the medical imaging apparatus, which has appropriate software for this purpose. In this way a high level of safety can be achieved for a patient when the feedback marking is projected. In particular, dazzling of a patient and/or in the case of an embodiment of the projection apparatus with a laser marking apparatus, burning the retina of patients can be advantageously prevented in this way.

In an advantageous development of the inventive method it can be provided that an examination position of the couch is calculated using the acquired position of the region to be examined of the object. The examination position of the couch is calculated by means of the computing unit of the medical imaging apparatus. The examination position comprises that position of the couch in respect of the scanner unit such that the region to be examined of the object, in particular of the patient, is arranged inside the isocenter of the medical imaging apparatus for the pending medical imaging examination. Simple and time-saving positioning of the region to be examined in the isocenter of the medical imaging apparatus is enabled in this way. In particular, the medical operator can advantageously be assisted in this way with the positioning of a patient inside the medical imaging apparatus and a positioning workflow can be simplified for inexperienced users.

In an advantageous development of the inventive method it can be provided that the couch is automatically moved into the examination position for an acquisition of medical imaging data of the region to be examined of the object. Simple and time-saving positioning of the region to be examined in the isocenter of the medical imaging apparatus can likewise be enabled in this connection. In an exemplary embodiment, the patient-positioning apparatus has for this purpose an appropriate horizontal adjustment unit and a position controller for positioning the couch in the examination position.

A medical imaging apparatus may include an acquisition apparatus adapted to acquire a position of a marker element, a projection apparatus adapted to project a feedback marking at the acquired position of the marker element, a camera and a computing unit (computer), wherein the medical imaging apparatus is adapted to carry out a method for acquiring a position of a region to be examined of an object for a medical imaging examination.

The medical imaging apparatus may be designed and/or adapted for acquiring medical and/or diagnostic image data of a patient and/or object.

The medical imaging apparatus may include a scanner unit (scanner) adapted to acquire the medical and/or diagnostic image data. The scanner unit surrounds a recording region of the medical imaging apparatus. The recording region may be cylindrical and embodied to receive the patient and/or the object, in particular the region to be examined of the patient and/or object, for a medical imaging examination.

For a medical imaging examination, the object, in particular der patient, in particular the region to be examined of the object, in particular of the patient, is positioned inside the recording region of the medical imaging apparatus. The Field of View (FOV) and/or an isocenter of the medical imaging apparatus may be arranged inside the recording region. The FOV may comprise an acquisition region of the medical imaging apparatus within which the conditions exist for acquiring medical image data inside the recording region, such as a homogeneous basic magnetic field in the case of an embodiment of the medical imaging apparatus as a magnetic resonance apparatus. The isocenter of the medical imaging apparatus may comprise the region and/or point inside the medical imaging apparatus which has the optimum and/or ideal conditions for acquiring medical image data. For example, in the case of an embodiment of the medical imaging apparatus as a magnetic resonance apparatus, the isocenter comprises the most homogeneous magnetic field region inside the magnetic resonance apparatus.

For moving and/or positioning the object and/or the patient, in particular the region to be examined of the object and/or of the patient, inside the recording region, the medical imaging apparatus comprises a patient-positioning apparatus. The patient-positioning apparatus has a couch with the couch being embodied to be movable relative to the scanner unit. In an exemplary embodiment, the couch is embodied to be movable in the longitudinal direction of the couch and/or in the longitudinal direction of the recording region inside the recording region in order to position the patient in an examination position inside the recording region for a medical imaging examination.

The medical imaging apparatus can comprise at least one camera embodied as a 3D camera. Alternatively, the medical imaging apparatus can also comprise at least two cameras embodied as 2D cameras.

The inventive medical imaging apparatus has the advantage that fast and exact marking of the region to be examined can be provided for a medical imaging examination. Furthermore, by way of the correct and/or exact marking of the marker element, and therewith of the region to be examined of the object, correct positioning of the region to be examined inside a patient-receiving region of a medical imaging apparatus can be achieved and therewith also incorrect positionings prevented. Medical imaging examinations can therewith also be carried out easily and quickly and repetitions of individual measurements owing to an incorrect positioning can be prevented. In particular, a medical operator can advantageously be assisted in this way in a workflow for positioning the region to be examined inside the isocenter.

A further advantage is that for inputting and/or acquiring the region to be examined of the object it is not necessary for a user input to be made at an input unit, for example touching a touch display, so hygiene standards can be maintained particularly easily during a medical imaging examination.

The advantages of the inventive medical imaging apparatus substantially correspond to the advantages of the inventive method for acquiring a position of a region to be examined of an object for a medical imaging examination, which have been stated above in detail. Features, advantages or alternative embodiments mentioned in this connection can likewise be transferred to the other claimed subject matters, and vice versa.

In an advantageous development of the inventive medical imaging apparatus it can be provided that the medical imaging apparatus comprises a scanner unit with a front side, wherein the acquisition apparatus and/or the projection apparatus is or are arranged in a front region in front of the front side of the scanner unit. The couch of the patient-positioning apparatus together with the object to be examined, in particular with the patient to be examined, are arranged during a preparation of the object, in particular of the patient, for the pending medical imaging examination in this front region in front of the front side of the scanner unit. This embodiment enables an advantageous arrangement of the acquisition apparatus and/or the projection apparatus with regard to acquiring the region to be examined for the medical imaging examination.

In an advantageous development of the inventive medical imaging apparatus it can be provided that the acquisition apparatus and/or the projection apparatus is or are permanently arranged on a ceiling of an examination space and/or at the front side of the scanner unit. The scanner unit of the medical imaging apparatus is arranged inside the examination space. A controller for controlling the scanner unit can likewise be arranged inside the examination space. In the case of an embodiment of the medical imaging apparatus as a magnetic resonance apparatus, the controller for controlling the magnetic resonance apparatus is arranged inside a control room which is separate from the examination space. In particular, the control room is shielded from the examination space with regard to radio-frequency radiation.

If the acquisition apparatus and/or the projection apparatus is arranged at the front side of the scanner unit of the medical imaging apparatus, then the acquisition apparatus and/or the projection apparatus may be arranged above an entry opening of the patient-receiving region of the scanner unit. This enables an advantageous orientation of a field of view and/or acquisition field of the acquisition apparatus and/or the projection apparatus onto the couch, in particular a reclining area of the couch, during a preparation of the patient.

The acquisition apparatus can orient an acquisition field and/or field of view of the acquisition apparatus in different directions, for example by rotating a camera of the acquisition apparatus, but acquisition apparatus is arranged and/or secured at a fixed point on the ceiling of the examination space. Similarly, the projection apparatus can also orient a field of view and/or projection field of the projection apparatus in different directions, for example by rotating a laser unit of the projection apparatus, but the arrangement and/or securing of the projection apparatus is at a fixed point on the ceiling of the examination space.

This embodiment of the disclosure enables a compact arrangement of the acquisition apparatus and/or the projection apparatus on the ceiling of the examination space. In particular, additional costs, which a mobile and/or movable arrangement of the acquisition apparatus and/or the projection apparatus on the ceiling of the examination space would entail, can also be saved in this way.

In an advantageous development of the inventive medical imaging apparatus it can be provided that the acquisition apparatus comprises the camera. A particularly compact and component-saving acquisition of the position of the region to be examined can be provided in this way. In an alternative embodiment the camera can also be embodied separately from the acquisition apparatus. The camera can comprise at least one 3D camera or also a plurality of 2D cameras. In particular, a 3D profile of the patient, in particular of the region to be examined of the patient, can be ascertained by means of at least two 2D cameras and therewith from at least two different 2D images of the region to be examined while exploiting stereoscopic effects.

In an advantageous development of the inventive medical imaging apparatus it can be provided that the medical imaging apparatus is embodied as a magnetic resonance apparatus. In particular in the case of magnetic resonance examinations which take up a relatively long time, an exact positioning is particularly important. The time a patient spends inside the recording region is thereby also reduced to the measuring time and repeated measurements due to incorrect positionings can thus be prevented.

The magnetic resonance apparatus may comprise a scanner unit, embodied as a magnetic unit, for acquiring the medical and/or diagnostic image data. The magnetic unit comprises a basic magnet, a gradient coil unit and a radio-frequency antenna unit here. The radio-frequency antenna unit is permanently arranged inside the magnetic unit here. The magnetic unit surrounds a recording region of the magnetic resonance apparatus. The recording region may be cylindrical and is embodied for a recording of the patient and/or of the object, in particular of the region to be examined of the patient and/or object, for a magnetic resonance examination.

The basic magnet is adapted to generate a homogeneous basic magnet field with a defined magnetic field strength, such as with a magnetic field strength of 0.55 T or 1.5 T or 3 T or 7 T, etc. In particular, the basic magnet is adapted to generate a strong, constant and homogeneous basic magnet field. The gradient coil unit is adapted to generate magnetic field gradients which are used for a spatial encoding during an imaging. The radio-frequency antenna unit is adapted to emit radio-frequency pulses and/or excitation pulses for generating magnetic resonance signals.

Furthermore, the disclosure starts from a computer program product which comprises a program and can be loaded directly in a memory of a programmable computing unit, with program means in order to carry out a method for acquiring a position of a region to be examined of an object for a medical imaging examination when the program is executed in the computing unit. The computer program potentially requires program means, for example libraries and auxiliary functions, in order to implement the corresponding embodiments of the method. The computer program can comprise software with a source code which still has to be compiled and linked or which only has to be interpreted, or an executable software code which just has to be loaded into a corresponding computing unit for execution.

The inventive computer program product can be loaded directly into a memory of a programmable computing unit and has program code means in order to execute an inventive method when the computer program product is executed in the computing unit. The computer program product can be a computer program or comprise a computer program. The inventive method can consequently be executed quickly, in an identically repeatable manner and robustly. The computer program product is configured in such a way that it can execute the inventive method steps by means of the computing unit. The computing unit must in each case have the requirements, such as an appropriate main memory, an appropriate graphics card or an appropriate logic unit, so the respective method steps can be efficiently executed. The computer program product is saved, for example, on a computer-readable medium or stored on a network or server from where it can be loaded into the processor of a local computing unit which can be directly connected to the magnetic resonance apparatus or can be embodied as part of it. Furthermore, items of control information of the computer program product can be saved on an electronically readable data carrier. The items of control information of the electronically readable data carrier can be embodied in such a way that they execute an inventive method when the data carrier is used in a computing unit. The computer program product can thus also constitute the electronically readable data carrier. Examples of electronically readable data carriers are a DVD, a magnetic tape, a hard disk or a USB stick on which electronically readable items of control information, in particular software (cf. above), is saved. When these items of control information (software) are read from the data carrier and saved in a controller and/or computing unit, all inventive embodiments of the above-described method can be carried out. The disclosure can thus also start from said computer-readable medium and/or said electronically readable data carrier.

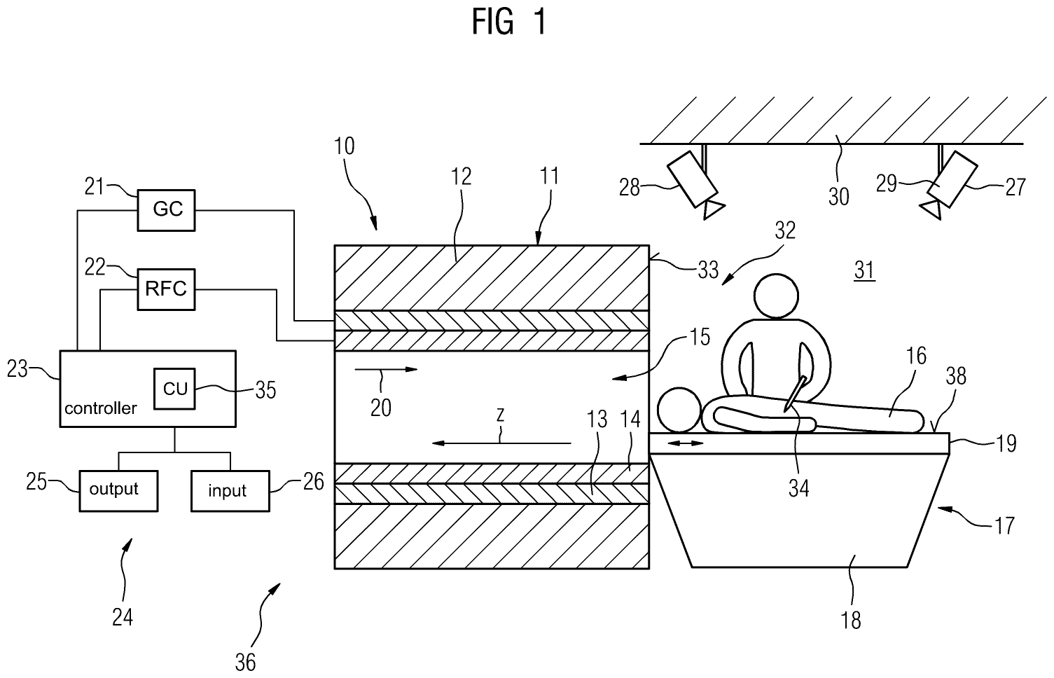

FIG. 1 schematically represents a medical imaging apparatus 36. In the present exemplary embodiment, the medical imaging apparatus 36 is formed by a magnetic resonance apparatus 10, with the present disclosure being explained, by way of example, on the basis of the magnetic resonance apparatus 10. The present disclosure is not limited to embodiment of the medical imaging apparatus 36 to a magnetic resonance apparatus 10, however, and further embodiments of the medical imaging apparatus 36 are conceivable at any time.

The magnetic resonance apparatus 10 may comprise a scanner unit (scanner), embodied as a magnetic unit 11, with a basic magnet 12, a gradient coil unit 13 and a radio-frequency antenna unit 14. In addition, the medical imaging apparatus 36, in particular the magnetic resonance apparatus 10, has a recording region 15 for recording an object and/or patient 16 for a magnetic resonance examination. In the present exemplary embodiment, the recording region 15 is cylindrical and cylindrically surrounded in a circumferential direction by the magnetic unit 11. Basically, a different embodiment of the recording region 15 is conceivable at any time, however.

The medical imaging apparatus 36, in particular the magnetic resonance apparatus 10, has a patient-supporting apparatus 17 for positioning the object and/or patient 16, in particular a region to be examined of the patient 16, inside the recording region 15. The patient-supporting apparatus 17 has a base unit (base) 18 and a couch 19 which can move in respect of the base unit 18. The couch 19 is embodied so it can move inside the recording region 15 for positioning of the object and/or patient 16, in particular the region to be examined of the patient 16. In particular, the couch 19 is movably mounted in the direction of a longitudinal extension of the recording region 15 and/or in the z-direction.

The basic magnet 12 of the magnetic unit 11 is adapted to generate a strong and, in particular, constant basic magnet field 20. The basic magnet 12 can be embodied, for example, as a superconducting basic magnet 12 or also as a permanent magnet. The gradient coil unit 13 of the magnetic unit 11 is embodied to generate magnetic field gradients which are used for spatial encoding during imaging. The gradient coil unit 13 is controlled by means of a gradient controller 21 of the magnetic resonance apparatus 10. The radio-frequency antenna unit 14 of the magnetic unit 11 is embodied to excite a polarization which is established in the basic magnetic field 20 generated by the basic magnet 12. The radio-frequency antenna unit 14 is controlled by a radio-frequency antenna controller 22 of the magnetic resonance apparatus 10 and irradiates radio-frequency magnetic resonance frequencies into the recording region 15 of the magnetic resonance apparatus 10.

The magnetic resonance apparatus 10 has a system controller 23 for controlling the basic magnet 12, the gradient controller 21 and for controlling the radio-frequency antenna controller 22. The system controller 23 centrally controls the magnetic resonance apparatus 10, such as carrying out a predetermined imaging gradient echo sequence, for instance. In addition, the system controller 23 comprises an evaluation unit (not represented) for an evaluation of medical image data which is acquired during the magnetic resonance examination. In an exemplary embodiment, the controller 23 (and/or one or more components therein, such as the computing unit 35) includes processing circuitry adapted to perform one or more operations or functions of the controller 23 (and/or respective components therein).

Furthermore, the medical imaging apparatus 36, in particular the magnetic resonance apparatus 10, comprises a user interface 24 which is connected to the system controller 23. Items of control information such as imaging parameters, as well as reconstructed magnetic resonance images can be displayed for a medical operator 33 on a representation unit 25, for example on at least one monitor, of the user interface 24. Furthermore, the user interface 24 has an input unit 26 by means of which items of information and/or parameters can be input by a medical operator 33 during a measuring process.

The medical imaging apparatus 36, in particular the magnetic resonance apparatus 10, has an acquisition apparatus 27, a projection apparatus (projector) 28 for positioning the patient 16. The acquisition apparatus 27 is adapted to acquire a position of a region to be examined of the object and/or of the patient 16 for a magnetic resonance examination. For this the acquisition apparatus 27 may have a camera, in particular a 3D camera 29. As an alternative to this the acquisition apparatus 27 can also comprise two or more 2D cameras. In the present exemplary embodiment, the acquisition apparatus 27 is permanently arranged on a ceiling 30 of an examination space 31 in which the magnetic unit 11 of the magnetic resonance apparatus 10 is arranged. The acquisition apparatus 27 is arranged in a front region 32 in front of a front side 33 of the magnetic unit 11 in order to acquire the patient 16 and/or the couch 19 before it is moved into the recording region 15. In an alternative embodiment of the disclosure the acquisition apparatus 27 can also be arranged at the front side 33 of the magnetic unit 11.

In order to acquire the region to be examined of the object, in particular of the patient 16, the medical operator may position a marker element 34 at the region to be examined of the object, in particular of the patient 16, which is identified and acquired by the acquisition apparatus 27. The marker element 34 can comprise, for example, a finger of the medical operator or also a hand-guided item, for example a marker bar.

The projection apparatus 28 may comprise a video projector and/or a laser marking unit which is adapted to project and/or display a feedback marking at the position of the marker element 34 acquired by the acquisition apparatus 27. The feedback marking is provided for the user, in particular the medical operator, in order to monitor a position of the region to be examined acquired by the acquisition apparatus 27. In the present exemplary embodiment, the projection apparatus 28 is permanently arranged on a ceiling 30 of the examination space 31. The projection apparatus 28 is arranged in the front region 32 in front of the front side 33 of the magnetic unit 11 in order to project a feedback marking onto the patient 16 before being moved into the recording region 15. In an alternative embodiment of the disclosure the projection apparatus 28 can also be arranged at the front side 33 of the magnetic unit 11.

The medical imaging apparatus 36, in particular the magnetic resonance apparatus 11, also has a 3D camera 29, which is embodied to acquire 3D data of the object positioned on the couch 19, in particular patient 16. In the present exemplary embodiment, the 3D camera 29 is incorporated by the acquisition apparatus 27.

For controlling the acquisition apparatus 27 and the projection apparatus 28 the medical imaging apparatus 36, in particular the magnetic resonance apparatus 10, has a computing unit (computer) 35. In the present exemplary embodiment, the computing unit 35 is integrated in the system controller 23. In an alternative embodiment the computing unit 35 can also be embodied separately from the system controller 23. In an exemplary embodiment, the computing unit 35 includes processing circuitry adapted to perform one or more operations or functions of the computing unit 35.

The represented medical imaging apparatus 36, in particular the magnetic resonance apparatus 10, can of course comprise further components which medical imaging apparatuses 36, in particular magnetic resonance apparatuses 10, customarily have. A general mode of operation of a medical imaging apparatus 36, in particular a magnetic resonance apparatus 10, is known to a person skilled in the art, moreover, so a detailed description of the further components will be omitted.

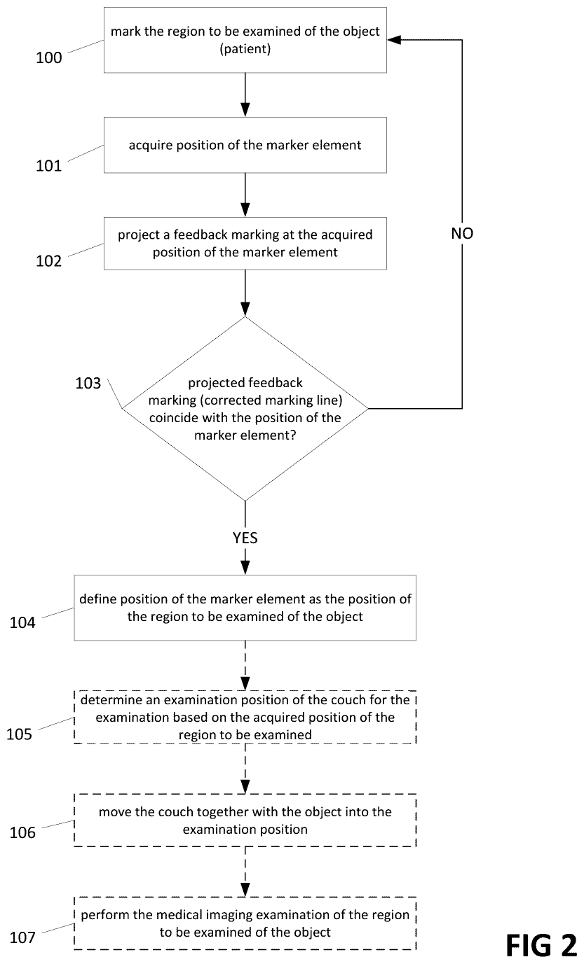

FIG. 2 represents a method for acquiring a position of a region to be examined of an object, in particular of a patient 16, for a medical imaging examination. The method is carried out by the computing unit 35 of the medical imaging apparatus 36, in particular the magnetic resonance apparatus 10, wherein by means of the computing unit 35 to control the individual method steps of the method for acquiring a position of a region to be examined of an object, in particular of a patient 16, for a medical imaging examination. The computing unit 35 has for this purpose appropriate control software and/or computer programs, which are stored in a memory of the computing unit 35. When the control software and/or the computer programs is/are executed by a processor of the computing unit 36 the individual method steps are actuated accordingly.

The inventive method is carried out during a preparation of the object, in particular of the patient 16, for a medical imaging examination. At the end of the preparation, before the object to be examined, in particular the patient 16 to be examined, is moved into the recording region 15 by means of the couch 19, in a first method step 100 the medical operator marks the region to be examined of the object, in particular of the patient 16. Marking occurs by way of manual positioning of the marker element 34 at the region to be examined of the object, in particular of the patient 16. The marker element 34 can comprise a finger of the medical operator or also a hand-guided marker element, such as a marker bar.

In a second method step 101 the position of the marker element 34 is acquired by means of the acquisition apparatus 27. The acquired position data of the acquisition apparatus 27 is directed to the computing unit 35 and is evaluated there and corresponding items of position information of the marker element 34 are ascertained.

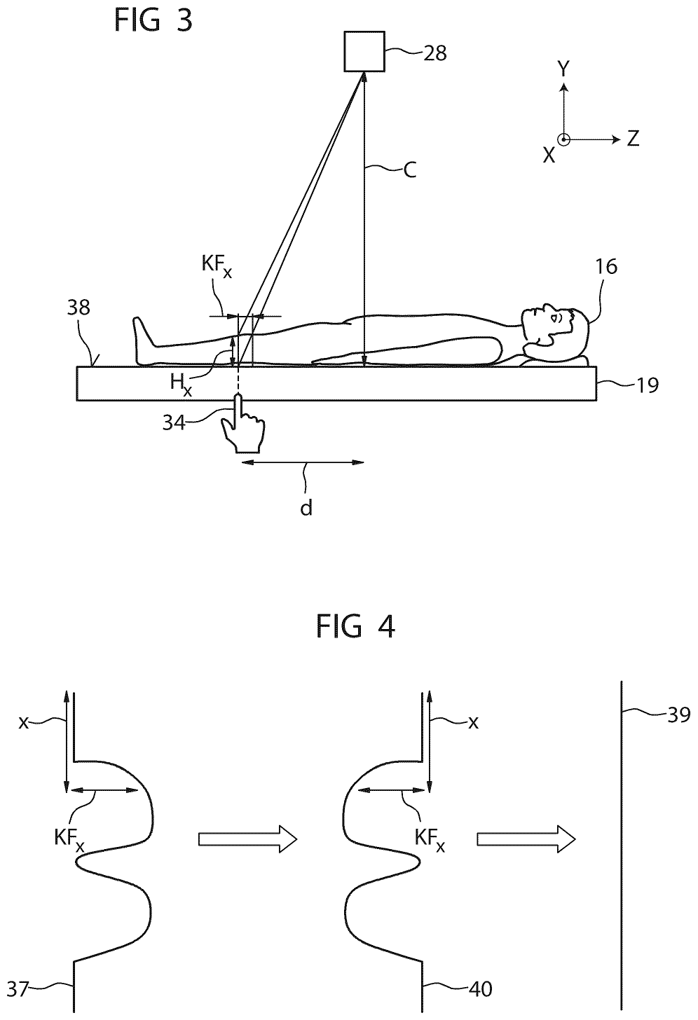

In a third method step 102, a feedback marking is projected at the acquired position of the marker element 34 by means of the projection apparatus 28. The feedback marking comprises a projected marking line 39 in the x-direction, with the feedback marking, in particular the marking line 39, being projected at the acquired position of the marker element onto the object, in particular onto the patient 16, by the projection apparatus 16. For this the projection apparatus 28 receives the items of position information of the marker element from the computing unit 35. The items of position information of the marking element, and therewith also the items of position information of the feedback marking, refer to a reclining area 38 of the couch 19. Since the object, in particular a patient 16, has a three-dimensional extent there is a distortion in the z-direction, which is dependent on a height of the profile of the patient 16 at the position of the marker element 34 in the z-direction (FIG. 3) when the feedback marking is projected. In order to eliminate this distortion when the feedback marking is projected, the projection apparatus 28 considers a correction factor KFₓ in the z-direction when the feedback marking, in particular the marking line 39, is generated and projected.

Three-dimensional data of the object, in particular of the patient 16, is acquired for determining and/or ascertaining the correction factor KFₓ. The 3D data is acquired by means of the 3D camera 29 of the acquisition apparatus 27. A 3D profile of the object, in particular of the patient 16, is created and/or determined by the computing unit 35 from the 3D data. A 3D profile of the object lying on the couch 19, in particular patient 16, is created and/or determined. The computing unit 35 also ascertains and/or determines a height Hₓ of the object, in particular of the patient 16, at the acquired position of the marker element 34 from the 3D profile. The computing unit 35 ascertains a height Hₓ of the object, in particular of the patient 16, from the 3D profile of the object, in particular of the patient 16, for each position in the x-direction at the position of the marking line 39.

The ascertained height Hₓ of the object, in particular of the patient 16, at the acquired position of the marker element 34 is included in the correction factor KFₓ. Apart from the ascertained height Hₓ of the object, in particular of the patient 16, the correction factor KFₓ also includes a spacing d of the marker element 34 from a position of the projection apparatus 28 in the z-direction and a height C of the projection apparatus 28 above the couch 19. The correction factor KFₓ is ascertained by the computing unit 35. The correction factor KFₓ is composed as follows:

$${KF}_{x} = \frac{d}{C} {H}_{x}$$

Here KFₓ is the correction factor and/or a correction value at the position x along the marking line 37, with the correction factor KFₓ an additive correction factor and/or correction value, which is deducted from an x-straight line at the x-position of the marker element 34. Hₓ is the height of the object, in particular of the patient 16, in the x-direction relative to the reclining area 38 of the couch 19 (FIGS. 3 and 4).

The correction factor KFₓ calculates for each x-position of the marking line without correction 37 a correction value and takes it into account when the feedback marking is projected, in particular when a corrected marking line 39 of the feedback marking is projected. FIG. 4 firstly represents a projection of the feedback marking, in particular the marking line 37, without correction factor KFₓ on a leg region of the patient 16, with the feedback marking, in particular the marking line 37, showing a dependency on the height Hₓ of the patient 16 along the x-direction and/or the marking line 37. The line 40 shows a feedback marking generated by the projection apparatus 28 before a projection onto the object, in the present exemplary embodiment onto the legs of the patient 16, in which the correction factor KFₓ is considered. This line 40 generated by the projection apparatus 28 is projected by the projection apparatus 28 as a corrected marking line 39 onto the object, in the present exemplary embodiment onto the legs of the patient 16. The correction factor KFₓ corrects the marking line 37 in such a way that it is projected independently of a height Hₓ of the patient 16 as a straight line, in particular as a straight and/or corrected marking line 39, onto the patient 16.

In addition, an eye area of the patient 16 is ascertained from the 3D data of a patient 16. This eye area of the patient 16 is excluded when the feedback marking, in particular the marking line 37, is subsequently projected from a projection region of the projection apparatus 28.

In a further, fourth method step 103, it is checked whether the projected feedback marking, in particular the corrected marking line 39, coincides with the position of the marker element 34 and therewith with the position of the region to be examined of the object, in particular of the patient 16. For example, after receiving the feedback marking, in particular the corrected marking line 39, if it does not coincide with a position of the marker element 34, and therewith of the region to be examined of the object, in particular of the patient 16, a manual repositioning of the marker element 34 communicates to the acquisition apparatus 27 that the acquired position was not correct. If the result of the check is that the projected feedback marking does not coincide with the position of the marker element 34, method step 101 of acquiring the position of the marker element 34 and method step 102 of projecting the feedback marking at the acquired position of the marker element 34 are repeated. Method step 101 of acquiring the position of the marker element 34 and method step 102 of projecting the feedback marking at the acquired position of the marker element 34 are repeated until the feedback marking coincides with the position of the marker element 34.

If, by contrast, the projected feedback marking does coincide with the position of the marker element 34, and therewith with the position of the region to be examined of the object, in particular of the patient 16, in this method step 103, this can be signaled to the acquisition apparatus 27, for example by retaining the position of the marker element 34 and/or a complete removal of the marker element 34 from the acquisition region of the acquisition apparatus 27. As soon as the position of the feedback marking coincides with the position of the marker element 34, and therewith of the region to be examined of the patient 16, for the user and the user signals to the acquisition apparatus 27 that the position is correct, the position of the marker element 34 is defined as the position of the region to be examined of the object, in particular of the patient 16, in a further, fifth method step 104.

In a further optional method step 105, an examination position of the couch 19 is calculated for the pending medical imaging examination, in particular magnetic resonance examination, using the acquired position of the region to be examined of the object, in particular of the patient 16. In a further optional method step 106, the couch 19 together with the object, in particular with the patient 16, is then moved into this examination position for acquiring medical imaging data, in particular magnetic resonance data, of the region to be examined. The region to be examined of the object, in particular of the patient 16, is located inside the recording region 15, in particular the isocenter, of the medical imaging apparatus 36, in particular the magnetic resonance apparatus 10, in this examination position. The medical imaging examination, in particular the magnetic resonance examination, of the region to be examined of the object, in particular of the patient 16, is then carried out in a further optional method step 107.

Although the disclosure has been illustrated and described in detail by the preferred exemplary embodiment, it is not limited by the disclosed examples and a person skilled in the art can derive other variations herefrom without departing from the scope of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for acquiring a position of a region to be examined of an object for a medical imaging examination, the method comprising:
manually positioning, by a medical operator, a marker element at the region to be examined of the object;
acquiring, by acquisition apparatus, a position of the marker element;
ascertaining a projection position of a feedback marking based on a correction factor in a z-direction;
projecting, by a projection apparatus, the feedback marking at the acquired position of the marker element;
repeating acquisition of the position of the marker element and of the projection position of the projection of the feedback marking until the projection position of the feedback marking coincides with the position of the marker element; and
in response to the projection position of the feedback marking coinciding with the position of the marker element, defining the position of the marker element as the position of the region to be examined of the object.

2. The method as claimed in claim 1, further comprising:
acquiring three-dimensional (3D) data of the object to determine the correction factor; and
determining a 3D profile of the object based on the 3D data.

3. The method as claimed in claim 2, further comprising determining a height of the object at the acquired position of the marker element is ascertained from the 3D profile.

4. The method as claimed in claim 2, wherein the feedback marking for defining the position of the region to be examined in the z-direction comprises a projected marking line in a x-direction on the object, wherein for each position in the x-direction of the marking line, a height of the object in a y-direction is ascertained from the 3D profile of the object.

5. The method as claimed in claim 4, wherein the z-direction is transverse to the x-direction, and the y-direction is transverse to both the z-direction and the x-direction.

6. The method as claimed in claim 2, wherein the correction factor in the z-direction comprises a height of the object at the acquired position of the marker element.

7. The method as claimed in claim 1, wherein the correction factor is based on a spacing of the marker element from a position of the projection apparatus in the z-direction and a height of the projection apparatus above a couch, in a x-direction transverse to the z-direction, supporting the object.

8. The method as claimed in claim 7, wherein the correction factor is further based on a height of the object, in the x-direction, at the acquired position of the marker element.

9. The method as claimed in claim 1, further comprising determining an eye area of the object based on three-dimensional (3D) data of object, the eye area being excluding from a projection region of the projection apparatus when the feedback marking is projected.

10. The method as claimed in claim 1, further comprising calculating an examination position of a couch supporting the object based on the acquired position of the region to be examined of the object.

11. The method as claimed in claim 10, further comprising automatically moving a couch supporting the object into the examination position for acquiring medical imaging data of the region to be examined of the object.

12. A non-transitory computer-readable storage medium with an executable program stored thereon, that when executed, instructs a processor to perform the method of claim 1.

13. A medical imaging apparatus comprising:
an acquisition apparatus adapted to acquire a position of a marker element that is positioned at a region to be examined of an examination subject;
a projection apparatus adapted to project a feedback marking at the acquired position of the marker element; and
a controller adapted to:
determine a projection position of the feedback marking based on a correction factor in a z-direction;
control the acquisition apparatus and the projection apparatus to repeatedly acquire the position of the marker element and the projection position of the project the feedback marking until the projection position of the feedback marking coincides with the position of the marker element; and
in response to the projection position of the feedback marking coinciding with the position of the marker element, define the position of the marker element as the position of the region to be examined of the examination subject.

14. The medical imaging apparatus as claimed in claim 13, further comprising a scanner, the acquisition apparatus and/or the projection apparatus being arranged in a front region in front of a front side of the scanner.

15. The medical imaging apparatus as claimed in claim 14, wherein the acquisition apparatus and/or the projection apparatus are permanently arranged on a ceiling of an examination space and/or on the front side of the scanner.

16. The medical imaging apparatus as claimed in claim 13, wherein the acquisition apparatus comprises a camera.

17. The medical imaging apparatus as claimed in claim 13, wherein the medical imaging apparatus is a magnetic resonance imaging apparatus.
